(19) United States
(12) Patent Application Publication
(10) Pub. No.: US 2009/0222099 A1
(43) Pub. Date: Sep. 3, 2009

(54) Self Centering Nucleus Implant

(75) Inventors: Mingyan Liu, Bourg la Reine (FR); Hai H. Trieu, Cordova, TN (US); Michael C. Sherman, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDICS, INC., Warsaw, IN (US)

(21) Appl. No.: 12/039,021

(22) Filed: Feb. 28, 2008

(51) Int. Cl. A61F 2/44

(52) U.S. Cl. 623/17.16; 623/17.12

(57) ABSTRACT

An intervertebral disc augmentation implant for implantation between a pair of vertebral bodies comprises an elastically deformable outer casing having at least one thickness dimension and a core member having isotropic material properties. The core member is entirely encased within the outer casing and has a height dimension along an axis defined by the pair of vertebral bodies. The modulus of elasticity of the core member is greater than a modulus of elasticity of the outer casing, and the height dimension of the core member is greater than the at least one thickness dimension of the outer casing.

### BACKGROUND

Within the spine, the intervertebral disc functions to stabilize and distribute forces between vertebral bodies. The intervertebral disc comprises a nucleus pulposus which is surrounded and confined by the annulus fibrosis.

Intervertebral discs are prone to injury and degeneration. For example, herniated discs typically occur when normal wear, or exceptional strain, causes a disc to rupture. Degenerative disc disease typically results from the normal aging process, in which the tissue gradually loses its natural water and elasticity, causing the degenerated disc to shrink and possibly rupture.

Intervertebral disc injuries and degeneration may be treated by fusion of adjacent vertebral bodies or by replacing the intervertebral disc with a prosthetic. To maintain as much of the natural tissue as possible, the nucleus pulposus may be supplemented or replaced while maintaining all or a portion of the annulus. A need exists for nucleus replacement and augmentation implants that will reduce the potential for implant migration within the annulus and/or expulsion from the annulus.

### SUMMARY

In one embodiment, an intervertebral disc augmentation implant for implantation between a pair of vertebral bodies comprises an elastically deformable outer casing having at least one thickness dimension and a core member having isotropic material properties. The core member is entirely encased within the outer casing and has a height dimension along an axis defined by the pair of vertebral bodies. The modulus of elasticity of the core member is greater than a modulus of elasticity of the outer casing, and the height dimension of the core member is greater than the at least one thickness dimension of the outer casing.

In another embodiment, a method of replacing a nucleus of an intervertebral disc located between a pair of vertebral bodies comprises accessing an annulus surrounding the nucleus and forming an opening in the annulus. The method further comprises inserting an intervertebral nucleus replacement implant. The implant comprises an elastically deformable outer casing having at least one thickness dimension and an isotropic core member entirely encased within the outer casing. The core member comprises a height dimension along an axis defined by the pair of vertebral bodies. A modulus of elasticity of the core member is greater than a modulus of elasticity of the outer casing. The height dimension of the core member is greater than the at least one thickness dimension of the outer casing.

In another embodiment, an implant for replacing at least a portion of a nucleus of an intervertebral disc between a pair of vertebral bodies comprises an elastically deformable outer casing having at least one thickness dimension and a non-composite core member having a height dimension along an axis defined through the pair of vertebral bodies. All surfaces of the core member are encased within and in direct contact with the outer casing, and a modulus of elasticity of the core member is greater than a modulus of elasticity of the outer casing.

Additional embodiments are included in the attached drawings and the description provided below.

### BRIEF DESCRIPTION OF THE DRAWINGS

FIG. 1 is a sagittal view of a section of a vertebral column.

FIG. 2 is a side cross sectional view of an implant with a core portion having a capsule shaped cross section.

FIG. 3 is a side cross sectional view of an implant with a core portion having an oval cross section.

FIG. 4 is a side cross sectional view of an implant with a core portion having an outer flange.

FIG. 5 is a side cross sectional view of an implant with a core portion having a circular cross section.

FIG. 6 is a top cross sectional view of an implant with a circular implant cross section and a circular core portion cross section.

FIG. 7 is a top cross sectional view of an implant with a capsule shaped cross section and a capsule shaped core portion cross section.

FIG. 8 is a top cross sectional view of an implant with a kidney shaped cross section and a kidney shaped core portion cross section.

FIG. 9 is a top cross sectional view of an implant with an oval shaped cross section and a circular core portion cross section.

FIG. 10 is a side cross sectional view of an implant under axial loading.

FIG. 11 is a side cross sectional view of an implant under offset loading.

### DETAILED DESCRIPTION

The present disclosure relates generally to devices and methods for relieving disc degeneration or injury, and more particularly, to devices and methods for augmenting a nucleus pulposus. For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

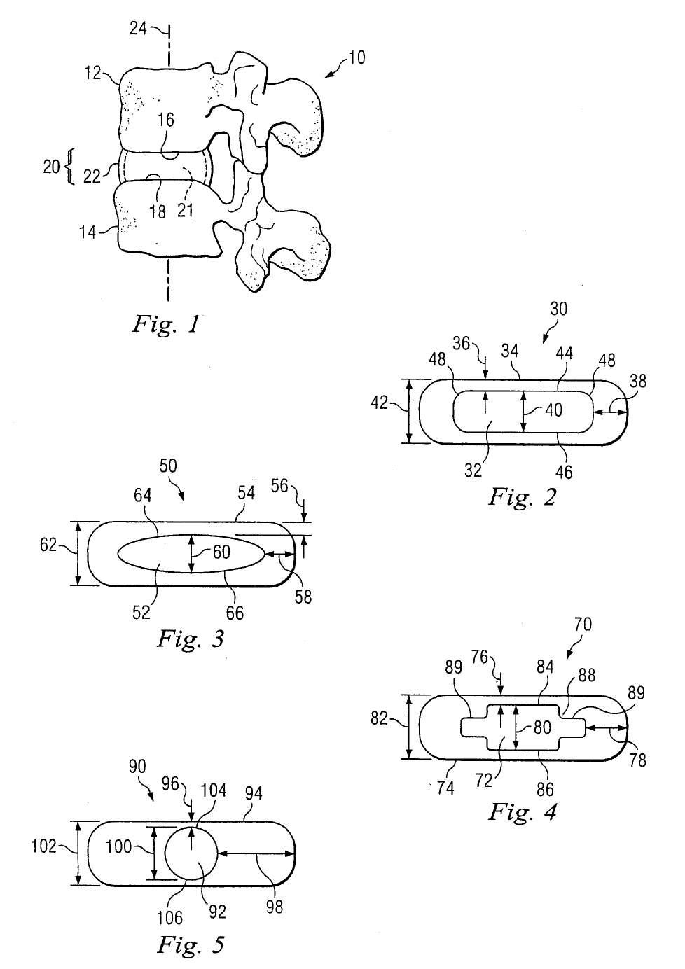

Referring first to FIG. 1, the reference numeral 10 refers to a vertebral joint section or a motion segment of a vertebral column. The joint section 10 includes adjacent vertebral bodies 12, 14. The vertebral bodies 12, 14 include endplates 16, 18, respectively. An intervertebral disc space 20 is located between the endplates 16, 18, and an annulus fibrosis 22 surrounds the space 20. In a healthy joint, the space 20 contains a nucleus pulposus 21. The nucleus pulposus 21 may degenerate with age, disease, or trauma. A central longitudinal axis 24 may extend through the vertebral joint 10.

Referring now to FIG. 2, a nucleus implant 30 may be used to augment the function and the existing tissue of the nucleus 21 or may be used to replace all or a portion of the nucleus 21. Thus, the implant 30 may fill all or a portion of the disc space 20 within the annulus 22. The implant 30 comprises a core portion 32 encapsulated within an outer casing 34.

The outer casing 34 is a skin-like layer which is softer and more elastically deformable than the core portion 32. Specifically, the outer casing 34 has a modulus of elasticity less than the modulus of elasticity of the core portion 32.

The outer casing 34 has a top thickness dimension 36 and a side thickness dimension 38. The thickness dimensions 36, 38 may be between 1 mm and 5 mm. The volume of the outer casing 34 may be between 5% and 50% of the total volume of the implant 30. Specifically, an outer casing volume between 20% and 30% of the total volume of the implant may be suitable.

The core portion 32 is harder and less elastically deformable than the outer casing 34. The core portion 32 may have a height 40 as measured along the axis 24. The height 40 may be greater than the thickness 36 and may even be greater than twice the thickness 36. The implant 30 may have an overall height 42 as measured along the axis 24. The thickness dimension 36 may be less than 25% of the of the implant height 42.

The core portion 32 has an upper surface 44, a lower surface 46, and outwardly radiused corners 48. In this embodiment, the upper and lower surfaces 44, 46 are generally flat, such that in the cross sectional side view, the core portion 32 has a capsule shaped profile.

Referring now to FIG. 3, a nucleus implant 50 may be used to augment the function and the existing tissue of the nucleus 21 or may be used to replace all or a portion of the nucleus 21. Thus, the implant 50 may fill all or a portion of the disc space 20 within the annulus 22. The implant 30 comprises a core portion 52 encapsulated within an outer casing 54.

The outer casing 54 is a skin-like layer which is softer and more elastically deformable than the core portion 52. Specifically, the outer casing 54 has a modulus of elasticity less than the modulus of elasticity of the core portion 52.

The outer casing 54 has a minimum top thickness dimension 56 and a minimum side thickness dimension 58. The thickness dimensions 56, 58 may be between 1 mm and 5 mm. The volume of the outer casing 54 may be between 5% and 50% of the total volume of the implant 50. Specifically, an outer casing volume between 20% and 30% of the total volume of the implant may be suitable.

The core portion 52 is harder and less elastically deformable than the outer casing 54. The core portion 52 may have a maximum height 60 as measured along the axis 24. The maximum height 60 may be greater than the minimum thickness 56 and may even be greater than twice the minimum thickness 56. The implant 50 may have an overall height 62 as measured along the axis 24. The minimum thickness dimension 56 may be less than 25% of the of the implant height 62.

The core portion 52 has an upper surface 64 and a lower surface 66. In this embodiment, the upper and lower surfaces 64, 66 are generally curved, such that in the cross sectional side view, the core portion 52 has an oval shaped profile.

Referring now to FIG. 4, a nucleus implant 70 may be used to augment the function and the existing tissue of the nucleus 21 or may be used to replace all or a portion of the nucleus 21. Thus, the implant 70 may fill all or a portion of the disc space 20 within the annulus 22. The implant 70 comprises a core portion 72 encapsulated within an outer casing 74.

The outer casing 74 is a skin-like layer which is softer and more elastically deformable than the core portion 72. Specifically, the outer casing 74 has a modulus of elasticity less than the modulus of elasticity of the core portion 72.

The outer casing 74 has a minimum top thickness dimension 76 and a minimum side thickness dimension 78. The thickness dimensions 76, 88 may be between 1 mm and 5 mm. The volume of the outer casing 74 may be between 5% and 50% of the total volume of the implant 70. Specifically, an outer casing volume between 20% and 30% of the total volume of the implant may be suitable.

The core portion 72 is harder and less elastically deformable than the outer casing 74. The core portion 72 may have a maximum height 80 as measured along the axis 24. The maximum height 80 may be greater than the minimum thickness 76 and may even be greater than twice the minimum thickness 76. The implant 70 may have an overall height 82 as measured along the axis 24. The minimum thickness dimension 76 may be less than 25% of the of the implant height 82.

The core portion 72 has an upper surface 84, a lower surface 86, inwardly radiused corners 88, and a perimeter flange 89. In this embodiment, the upper and lower surfaces 84, 86 are generally flat and intersect with the flange 89 at the inwardly radiused corners 88.

Referring now to FIG. 5, a nucleus implant 90 may be used to augment the function and the existing tissue of the nucleus 21 or may be used to replace all or a portion of the nucleus 21. Thus, the implant 90 may fill all or a portion of the disc space 20 within the annulus 22. The implant 90 comprises a core portion 92 encapsulated within an outer casing 94.

The outer casing 94 is a skin-like layer which is softer and more elastically deformable than the core portion 92. Specifically, the outer casing 94 has a modulus of elasticity less than the modulus of elasticity of the core portion 92.

The outer casing 94 has a minimum top thickness dimension 96 and a minimum side thickness dimension 98. The thickness dimension 96 may be between 1 mm and 5 mm. The volume of the outer casing 94 may be between 5% and 50% of the total volume of the implant 90. Specifically, an outer casing volume between 20% and 30% of the total volume of the implant may be suitable.

The core portion 92 is harder and less elastically deformable than the outer casing 94. The core portion 92 may have a maximum height 100 as measured along the axis 24. The maximum height 100 may be greater than the minimum thickness 96 and may even be greater than twice the minimum thickness 96. The implant 90 may have an overall height 102 as measured along the axis 24. The minimum thickness dimension 96 may be less than 25% of the of the implant height 102.

The core portion 92 has an upper surface 104 and a lower surface 106. In this embodiment, the upper and lower surfaces 104, 106 are generally curved, such that in the cross sectional side view, the core portion 92 has a circular profile.

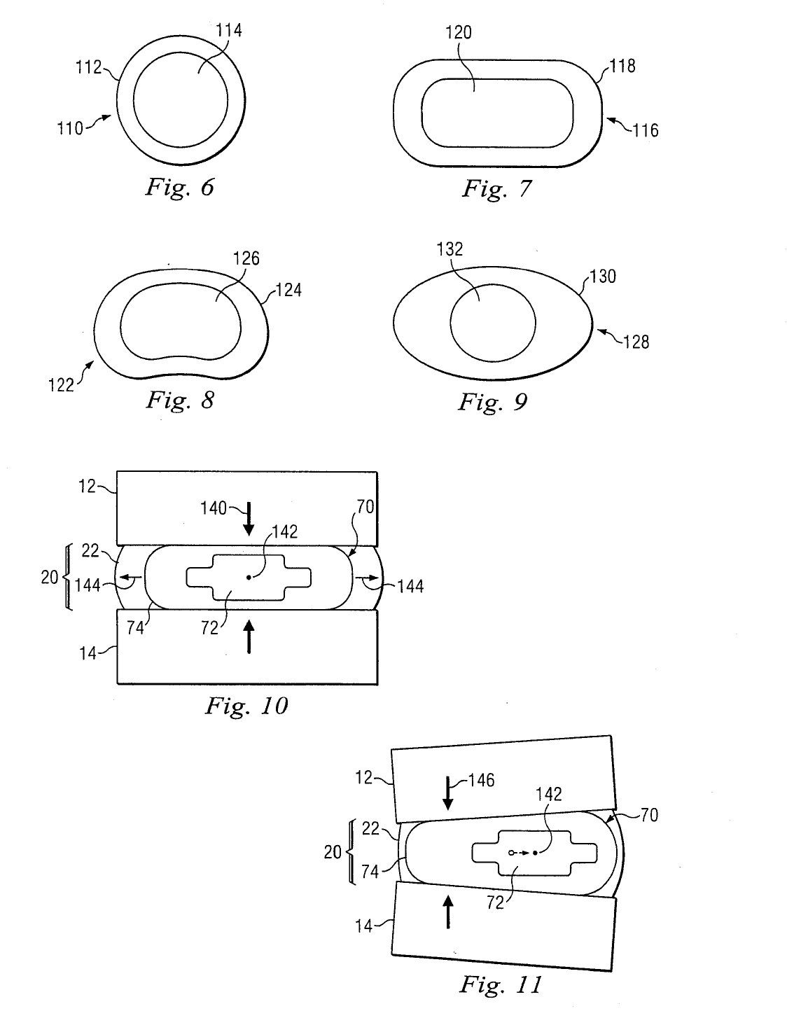

Referring now to FIG. 6, a nucleus implant 110 may have a side cross-sectional view the same as or similar to any of the implants 30, 50, 70, 90 described above. The implant 110 has an outer casing 112 surrounding a core portion 114. In this embodiment, a top cross- sectional view of the implant 110 is circular with a circular core portion 114.

Referring now to FIG. 7, a nucleus implant 116 may have a side cross-sectional view the same as or similar to any of the implants 30, 50, 70, 90 described above. The implant 116 has an outer casing 118 surrounding a core portion 120. In this embodiment, a top cross-sectional view of the implant 116 is capsule shaped with a capsule shaped core portion 120.

Referring now to FIG. 8, a nucleus implant 122 may have a side cross-sectional view the same as or similar to any of the implants 30, 50, 70, 90 described above. The implant 122 has an outer casing 124 surrounding a core portion 126. In this embodiment, a top cross-sectional view of the implant 122 is kidney shaped with a kidney shaped core portion 120.

Referring now to FIG. 9, a nucleus implant 128 may have a side cross-sectional view the same as or similar to any of the implants 30, 50, 70, 90 described above. The implant 128 has an outer casing 130 surrounding a core portion 132. In this embodiment, a top cross-sectional view of the implant 128 is oval shaped with a circular core portion 132. Thus, a core portion may have a different shape than the overall implant.

The overall implants and the core portions described above may assume any of a variety of three-dimensional shapes including spherical, elliptoid, boomerang, Saturn-like, disc, capsule, kidney, or cylindrical.

Any of the core portions in the embodiments described above may be uniform, non-composite structures and may have isotropic material properties throughout the core portion. Composite structures, such as layered structures, having anisotropic material properties may also be suitable. All surfaces of the core portion may be in direct contact with the outer casing. However, in composite structures, only outer edges of the inner layers may be in contact with the casing. The core portions described above may be formed of polymers such as ultra-high molecular weight polyethylene (UHMWPE), polyurethane, silicone-polyurethane copolymers, polyetheretherketone, or polymethylmethacrylate. Suitable metals may include cobalt-chrome alloys, titanium, titanium alloys, stainless steel, or titanium nickel alloys. Suitable ceramics may include alumina, zirconia, polycrystalline diamond compact, or pyrolitic carbon. In embodiments in which the core portion is formed from radiolucent materials, a radiocontrast marker or material such as barium sulfate, tungsten, tantalum, or titanium may be added to the core portion for purposes of viewing the implant with imaging equipment.

The outer casings may be formed of polyurethane, silicone, silicone polyurethane copolymers, polyolefins, such as polyisobutylene rubber and polyisoprene rubber, neoprene rubber, nitrile rubber, vulcanized rubber and combinations thereof. Any of the outer casings in the embodiments described above may be uniform, non-uniform or varying in thickness. For example in FIG. 4 above, the thickness of the casing 74 is greater in the peripheral area near the radiused corners 88 than in the more central region over the upper surface 84. The casings described in the embodiments above may reduce the contact stress between the core portion of the implant and the adjacent tissue as the spinal joint undergoes flexion-extension and lateral bending motion. The deformable properties of the casings may also serve to reduce the potential for implant migration or expulsion through an opening in the annulus.

In one exemplary embodiment, the core portion may be formed of UHMWPE with the outer casing formed of silicone having a durometer hardness of 60 Shore A. In another exemplary embodiment, the core portion may be formed of 80 Shore A BIONATE® polycarbonate-urethane with the outer casing formed of 50 Shore A silicone. In another exemplary embodiment, the core portion may be formed of 80 Shore A PURSIL silicone-polyetherurethane with the outer casing formed of 50 Shore A elastomeric polyurethane. All durometer hardness values are approximate. The core portion, for example, may have a hardness greater than the exemplary values. The outer casing, for example, may have a hardness lower than the exemplary values.

Prior to positioning any of the implants described above in the intervertebral disc space 20, an incision may be made in the annulus fibrosis or an existing annulus defect may be identified. The annulus 22 may be accessed through a posterior, lateral, anterior, or any other suitable approach. In one embodiment, a guide wire or other small instrument may be used to make the initial hole. If necessary, successively larger holes are cut from an initially small puncture. The hole (also called an aperture, an opening, or a portal, for example) may be as small as possible to minimize expulsion of the material through the hole after the surgery is complete. Also if necessary, a dilator may be used to dilate the hole, making it large enough to deliver the implant to replace or augment the disc nucleus. The dilator may stretch the hole temporarily and avoid tearing so that the hole can return back to its undilated size after the instrument is removed. Although some tearing or permanent stretching may occur, the dilation may be accomplished in a manner that allows the hole to return to a size smaller than its dilated size after the surgery is complete. In alternative embodiments, portions of the annulus 22 may be resected to allow passage of the implants.

Through the annulus opening, all or a portion of the natural nucleus pulposus may be removed. Any of a variety of tools may be used to prepare the disc space, including specialized pituitary rongeurs and curettes for reaching the margins of the nucleus pulposus. Ring curettes may be used to scape abrasions from the vertebral endplates as necessary. Using these instruments, a centralized, symmetrical space large enough to accept the implant footprint may be prepared in the disc space. It is understood that the natural nucleus pulposus need not be removed, but rather, an implant of the type described above may be used in cooperation with existing nucleus tissue to compensate for deficiencies in the existing tissue. The disc space may then be distracted to a desired level by distractors or other devices known to the skilled artisan for such purposes. After preparing the disc space 20 and/or annulus 22 for receiving the implant, the implant may be delivered into the intervertebral disc space using any of a variety of techniques known in the art.

Referring now to FIG. 10, the implant 70 may be installed within the disc space 20 using a technique such as that described above. In this embodiment, the implant 70 is subjected to an axial load 140 equally distributed about a center of mass 142. The relatively thin outer casing 74 above and below the center of mass 142 may increase the axial load bearing capability of the implant 70. The thin outer casing 74 allows the load 140 to be transmitted almost directly to the more rigid core portion 72 which is able to provide greater support. Under axial loading 140, the outer casing 74 may deform, expanding radially as shown by arrows 144.

Referring now to FIG. 11, the implant 70 may be subjected to a off-set loads 146 under flexion-extension or lateral bending motions. Under these types of motions, the thicker outer casing 74 near the peripheral portion of the implant 70 may reduce the contact stress between the vertebral bodies 12, 14 and the core portion 72, increasing the stress distribution. Under the off-set load 146, the center of mass 142 of the core portion 72 may shift away from the load 146. When the spinal joint 10 is returned to alignment, the elastic outer casing 74 may return to its original configuration and thereby cause the core portion 72 to return to its original position within the casing 74. In this way the implant 70 may have self-centering qualities. The deformable nature of the casing 74 and the self-centering nature of the implant 70 may reduce the likelihood that the entire implant will migrate or become expelled from the annulus 22.

As used throughout this description, the terms “modulus” and “modulus of elasticity” are broadly used to refer to physical material properties such as hardness or elasticity. High modulus materials are relatively hard or stiff, and low modulus materials are relatively soft and resilient.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “anterior,” “posterior,” “superior,” “inferior,” “upper,” and “lower” are for illustrative purposes only and can be varied within the scope of the disclosure. In the claims, means-plus-function clauses are intended to cover the elements described herein as performing the recited function and not only structural equivalents, but also equivalent elements.

What is claimed is:

1. An intervertebral disc augmentation implant for implantation between a pair of vertebral bodies comprising:
an elastically deformable outer casing having at least one thickness dimension; and
a core member having isotropic material properties and entirely encased within the outer casing and having a height dimension along an axis defined by the pair of vertebral bodies,
wherein a modulus of elasticity of the core member is greater than a modulus of elasticity of the outer casing and further wherein the height dimension of the core member is greater than the at least one thickness dimension of the outer casing.

2. The implant of claim 1 wherein the height dimension is at least twice as great as the at least one thickness dimension of the outer casing.

3. The implant of claim 1 wherein the core member has a curved upper surface.

4. The implant of claim 1 wherein the core member has a flat upper surface.

5. The implant of claim 1 wherein the core member is substantially spherical.

6. The implant of claim 1 wherein the core member is cylindrical.

7. The implant of claim 1 wherein the modulus of elasticity of the outer casing is no more than 75% of the modulus of elasticity of the core member.

8. The implant of claim 1 wherein the implant has an overall height dimension along an axis defined by the pair of vertebral bodies and wherein the at least one thickness dimension of the outer casing is not greater than 25% of the overall height dimension of the implant.

9. The implant of claim 1 wherein the at least one thickness dimension is between 1 mm and 5 mm.

10. The implant of claim 1 wherein the outer casing has a total casing volume and the implant has a total implant volume and wherein the total casing volume is between 5% and 50% of the total implant volume.

11. The implant of claim 10 wherein the total casing volume is between 10% and 40% of the total implant volume.

12. The implant of claim 10 wherein the total casing volume is between 20% and 30% of the total implant volume.

13. The implant of claim 1 wherein the outer casing comprises silicone.

14. The implant of claim 1 wherein the outer casing comprises polyurethane.

15. The implant of claim 1 wherein the outer casing comprises polyolefin rubber.

16. The implant of claim 1 wherein the core member comprises a radiocontrast material.

17. The implant of claim 1 wherein the at least one thickness dimension of the outer casing includes a peripheral thickness dimension and a central thickness dimension and further wherein the peripheral thickness dimension is greater than the central thickness dimension.

18. The implant of claim 1 wherein the core member comprises ultra high molecular weight polyethylene and the outer casing comprises silicone, and wherein the outer casing has a hardness equal or less than 60 Shore A.

19. The implant of claim 1 wherein the core member comprises polyurethane and has a hardness greater than or equal to 80 Shore A and wherein the outer casing comprises silicone and has a hardness less than or equal to 50 Shore A.

20. The implant of claim 1 wherein the core member comprises a silicone polyurethane copolymer and has a hardness greater than or equal to 80 Shore A and wherein the outer casing comprises polyurethane and has a hardness less than or equal to 50 Shore A.

21. The implant of claim 1 wherein the core member comprises at least one radiused corner.

22. The implant of claim 21 wherein the at least one radiused corner is an outwardly radiused corner.

23. The implant of claim 21 wherein the at least one radiused corner is an inwardly radiused corner.

24. A method of replacing a nucleus of an intervertebral disc located between a pair of vertebral bodies, the method comprising:
accessing an annulus surrounding the nucleus;
forming an opening in the annulus; and
inserting an intervertebral nucleus replacement implant wherein the implant comprises
an elastically deformable outer casing having at least one thickness dimension; and
an isotropic core member entirely encased within the outer casing and having a height dimension along an axis defined by the pair of vertebral bodies,
wherein a modulus of elasticity of the core member is greater than a modulus of elasticity of the outer casing and further wherein the height dimension of the core member is greater than the at least one thickness dimension of the outer casing.

25. The method of claim 24 further comprising:
removing at least a portion of the nucleus through the opening in the annulus.

26. The method of claim 24 wherein the step of inserting further comprises placing the implant in contact with at least a portion of the nucleus.

27. An implant for replacing at least a portion of a nucleus of an intervertebral disc between a pair of vertebral bodies, the implant comprising:
an elastically deformable outer casing having at least one thickness dimension; and
a non-composite core member having a height dimension along an axis defined through the pair of vertebral bodies,
wherein all surfaces of the core member are encased within and in direct contact with the outer casing and further wherein a modulus of elasticity of the core member is greater than a modulus of elasticity of the outer casing.

28. The implant of claim 27 wherein the modulus of elasticity of the outer casing is less than 75% of the modulus of elasticity of the core member.

29. The implant of claim 27 wherein the outer casing comprises silicone.

30. The implant of claim 27 wherein the outer casing comprises polyurethane.

31. The implant of claim 27 wherein the at least one thickness dimension of the outer casing includes a peripheral thickness greater than a central thickness.

32. The implant of claim 27 wherein the core member comprises a curved upper surface.

33. The implant of claim 27 wherein the core member comprises an outer flange.
